(19) United States
(12) Patent Application Publication
(10) Pub. No.: US 2025/0174032 A1
(43) Pub. Date: May 29, 2025

(54) CELL IMAGE PROCESSING SYSTEM AND CELL IMAGE PROCESSING METHOD

(71) Applicant: CANON KABUSHIKI KAISHA, Tokyo (JP)

(72) Inventors: SHUHEI TOBA, Kanagawa (JP); ICHIRO HARADA, Kanagawa (JP)

(21) Appl. No.: 18/960,459

(22) Filed: Nov. 26, 2024

(30) Foreign Application Priority Data
Nov. 29, 2023 (JP) 2023-201982

(51) Int. Cl. G06V 20/69; G06T 7/62; G06V 10/22; G06V 10/26; G06V 10/44

(52) U.S. Cl. CPC G06V 20/698; G06T 7/62; G06V 10/23; G06V 10/26; G06V 10/44; G06T 2207/30024

(57) ABSTRACT

A cell image processing system includes an image acquisition unit configured to acquire a cell image including a plurality of cells as a subject, a cell region extraction unit configured to extract a plurality of cell regions from the cell image, a cell information acquisition unit configured to acquire characteristic information including morphological information about each of the cell regions and pixel information about each of the cell regions, a representative cell determination unit configured to determine a representative cell region from among the plurality of extracted cell regions based on at least one type of information constituting the characteristic information, and a display control unit configured to cause a display unit to display image information about a cell region corresponding to the representative cell, positional information corresponding to the representative cell region, and characteristic information about a cell region corresponding to the representative cell.

### BACKGROUND OF THE DISCLOSURE

### Field of the Disclosure

The present disclosure relates to a cell image processing system and a cell image processing method.

### Description of the Related Art

An operation in which cell analysis data obtained by cell image analysis is acquired and the acquired analysis data is checked and analyzed by a user is often carried out. Specifically, an analysis data distribution is visualized in a format such as a scatter diagram so that the user can recognize the overall image of the analysis data distribution, thereby quantitatively recognizing the tendency of a cell data group. Further, an operation in which the user checks each cell data in the visualized data distribution while observing a state of each cell on an observation image is also carried out, to thereby check the state of each living cell while recognizing an overview of the tendency of quantitative data.

In cell image analysis software for supporting such operations, the user sequentially picks up cells of interest while checking the cells in the observation image. Further, cropped images including the cells and cell data can be listed.

Japanese Patent Application Laid-Open No. 2016-90234 discusses an image processing apparatus that extracts a feature value from a slice image and causes a display unit to display a feature value image including information about the extracted feature value in such a manner that the feature value image is superimposed on the slice image.

However, in many cases, a large number of cells, for example, several thousands or more of cells are treated as cell data to be analyzed. In such cases, it is difficult for a user to find out a primary cell among the large number of cells by a manual operation. The user needs to select the slice image, which increases a load on the user.

### SUMMARY OF THE DISCLOSURE

According to an aspect of the present disclosure, a cell image processing system includes an image acquisition unit configured to acquire a cell image of a sample including a plurality of cells as a subject, a cell region extraction unit configured to extract a plurality of cell regions respectively corresponding to cells from the cell image, a cell information acquisition unit configured to acquire characteristic information including morphological information about each of the cell regions and pixel information about each of the cell regions, a representative cell determination unit configured to determine a representative cell region from among the plurality of extracted cell regions based on at least one type of information constituting the characteristic information, and a display control unit configured to cause a display unit to display image information about a cell region corresponding to the representative cell, positional information corresponding to the representative cell region, and characteristic information about a cell region corresponding to the representative cell.

According to another aspect of the present disclosure, a cell image processing method includes acquiring a cell image of a sample including a plurality of cells as a subject, extracting a plurality of cell regions respectively corresponding to cells from the cell image, acquiring characteristic information including morphological information about each of the cell regions and pixel information about each of the cell regions, determining a representative cell region from among the plurality of extracted cell regions based on at least one type of information constituting the characteristic information, and controlling a display unit to display image information about a cell region corresponding to the representative cell, positional information corresponding to the representative cell region, and characteristic information about a cell region corresponding to the representative cell.

Further features of the present disclosure will become apparent from the following description of exemplary embodiments with reference to the attached drawings.

### BRIEF DESCRIPTION OF THE DRAWINGS

FIG. 1 is a block diagram illustrating functional blocks of a cell image processing system according to an exemplary embodiment of the present disclosure.

FIG. 2 is a flowchart illustrating a cell image processing method according to an exemplary embodiment of the present disclosure.

FIG. 3 is a flowchart illustrating an example of processing for acquiring cell data in step S201 according to an exemplary embodiment of the present disclosure.

FIG. 4 illustrates an example of a defocused image acquired in step S301 according to an exemplary embodiment of the present disclosure.

FIG. 5 illustrates an example of an autofluorescence image acquired in step S302 according to an exemplary embodiment of the present disclosure.

FIG. 6 illustrates an example of a mask image generated in step S303 according to an exemplary embodiment of the present disclosure.

FIG. 7 illustrates table information as an example of cell data acquired in step S201 according to an exemplary embodiment of the present disclosure.

FIG. 8 is a conceptual diagram illustrating a method for determining a representative cell in step S202 according to an exemplary embodiment of the present disclosure.

FIG. 9 is a conceptual diagram illustrating a method for determining a representative cell according to a fifth modified example of an exemplary embodiment of the present disclosure.

FIG. 10 illustrates an example of a graphical user interface (GUI) screen displaying information about representative cells in step S203 according to an exemplary embodiment of the present disclosure.

FIG. 11 illustrates an example of the GUI screen displaying information about representative cells according to a sixth modified example of an exemplary embodiment of the present disclosure.

FIG. 12 is a block diagram illustrating functional blocks of a cell image processing system according to an exemplary embodiment of the present disclosure.

FIG. 13 is a flowchart illustrating cell image processing according to an exemplary embodiment of the present disclosure.

FIG. 14 illustrates an example of the GUI screen displaying information about representative cells in step S1304 according to an exemplary embodiment of the present disclosure.

FIG. 15 is a block diagram illustrating an example of an information processing system configured to execute a program according to an exemplary embodiment of the present disclosure.

### DESCRIPTION OF THE EMBODIMENTS

Exemplary embodiments of the present disclosure will be described in detail below with reference to the accompanying drawings. The following exemplary embodiments are not intended to limit the claimed disclosure. A plurality of features is described in the exemplary embodiments, but not all such features are required for a disclosure, and such a plurality of features may be combined as appropriate. Furthermore, in the attached drawings, identical or like components are denoted by the same reference numeral, and redundant description thereof is omitted.

### <Cell Image Processing System>

A cell image processing system according to a first exemplary embodiment of the present disclosure will be described below.

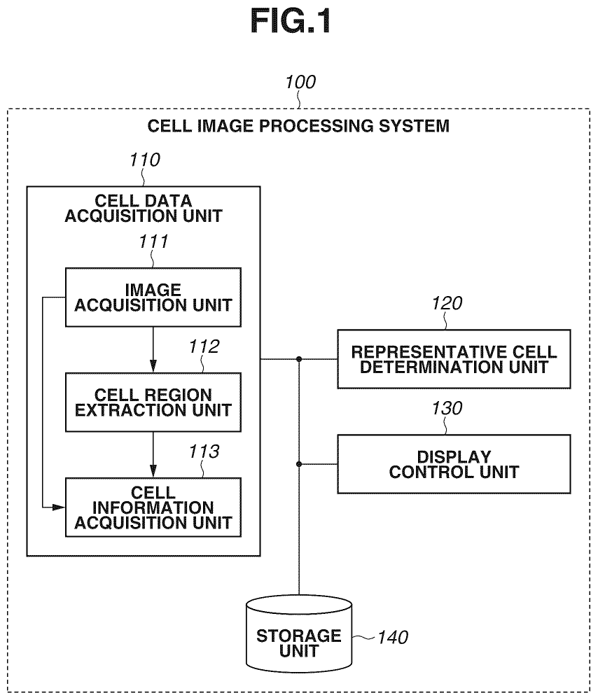

FIG. 1 is a block diagram illustrating functional blocks of the cell image processing system according to the first exemplary embodiment. A cell image processing system 100 includes a cell data acquisition unit 110, a representative cell determination unit 120, a display control unit 130, and a storage unit 140. The cell data acquisition unit 110 includes an image acquisition unit 111, a cell region extraction unit 112, and a cell information acquisition unit 113. While FIG. 1 illustrates each unit constituting the cell image processing system 100 as an integrated device, some of the units may be configured as an external device (including an object on a cloud).

### (Cell Data Acquisition Unit)

The cell data acquisition unit 110 acquires a cell image and a cell data group obtained by performing an image analysis on the cell image. The cell data group is a data group that is related to characteristic information about each cell region, including morphological information about the cell region and pixel information about cells, and is obtained by performing an image analysis on each cell region included in the cell image.

In the present exemplary embodiment, positional information about each cell region includes at least one of (i) a group of contour coordinates indicating a boundary between cell regions in the cell image, and (ii) coordinates indicating a position of center of gravity of each cell region in the cell image. The positional information about each cell region according to the present exemplary embodiment is not particularly limited as long as the positional information indicates the position of the cell region. Examples of the positional information include information about coordinates of the center of gravity of each cell region in the cell image and information about a group of contour coordinates indicating a boundary between cell regions.

In the present exemplary embodiment, morphological information about each cell region includes at least one of the area of each cell region, the diameter (radius) of each cell region, the circularity of each cell region, and the perimeter of the contour of each cell region.

In the present exemplary embodiment, pixel information about each cell region includes at least one of an average value of pixel (values) in each cell region, an integrated value of pixel values in each cell region, and a standard deviation of pixel values in each cell region.

### (Image Acquisition Unit)

The image acquisition unit 111 acquires a cell image (cell image data) of at least one of a bright-field image of each cell, a fluorescence image of each cell, and a phase difference image of each cell. The bright-field image is, for example, an image indicating the morphology of each cell, such as a bright-field image of each cell obtained at a focal point position, or a bright-field image captured by shifting the focal point position in an optical axis direction by a predetermined distance (this image is hereinafter referred to as a defocused image). The defocused image has a characteristic that a contour portion of a phase object including a cell is highlighted with higher pixel values or lower pixel values.

The fluorescence image is, for example, an image obtained by capturing an image of each cell to which light with a specific wavelength is applied from an excitation light source to thereby capture the autofluorescence of each cell (this image is hereinafter referred to as an autofluorescence image). The phase difference image is obtained by capturing an image of each cell by a phase difference microscope.

The cell image (cell image data) may be image data preliminarily stored in the storage unit 140, or may be acquired from an external storage region such as a hard disk drive (HDD) or a cloud storage. The cell image processing system 100 according to the present exemplary embodiment can be connected to a cell image observation apparatus (not illustrated) via a communication interface (I/F) to acquire an image captured by the cell image observation apparatus.

### (Cell Region Extraction Unit)

The cell region extraction unit 112 extracts a cell region from the cell image acquired by the image acquisition unit 111. Extracting a cell region means that the morphology of each of the cells that are discretely present within a cell observation field of view is extracted and acquired, or that each of the cell groups that are present in close contact with each other or in an aggregated state and the morphology of the entire aggregate are extracted and acquired. Software to be used for extracting each cell region is not particularly limited and known software can be used.

### (Cell Information Acquisition Unit)

The cell information acquisition unit 113 acquires characteristic information including morphological information about the cell region and pixel information about the cell region based on the cell image acquired by the image acquisition unit 111 and the cell region extracted by the cell region extraction unit 112.

### (Representative Cell Determination Unit)

The representative cell determination unit 120 determines a representative cell (primary cell) constituting the cell data group based on cell data acquired by the cell data acquisition unit 110. Specifically, the representative cell determination unit 120 determines a representative cell region from among a plurality of extracted cell regions based on at least one type of information constituting the characteristic information.

The representative cell determination unit 120 according to the present exemplary embodiment may extract a plurality of subsets from a data group formed of the characteristic information about the plurality of cell regions, and may determine the representative cell region from each of the plurality of subsets. An example of the subset extraction method is as follows. That is, the method includes (1) calculating a probability of occurrence of data on each cell region based on at least one type of information constituting characteristic information, and (2) extracting, as a subset, a data group corresponding to each of a plurality of predetermined occurrence probability ranges from the data group formed of the characteristic information about the plurality of cell regions. Another example of the subset extraction method is as follows. That is, the method includes (a) calculating a distance of data on each cell region from a center of gravity of data on all the cell regions based on at least one type of information constituting the characteristic information, and (2) extracting, as a subset, a data group corresponding to each of a plurality of predetermined distance ranges from the data group formed of the characteristic information about the plurality of cell regions. Still another example of the subset extraction method is as follows. That is, the method includes (i) dividing the data group constituting the subsets into a plurality of clusters by unsupervised clustering, and (ii) determining a cell region corresponding to data closest to a position of center of gravity of the data group to be the representative cell region in each of data groups respectively corresponding to the clusters. A process for determining the representative cell will be described in detail below.

### (Display Control Unit)

The display control unit 130 displays information about a representative cell region determined by the representative cell determination unit 120. Specifically, the display control unit 130 causes a display unit to display image information about a cell region corresponding to the representative cell, positional information corresponding to the representative cell region, and characteristic information about a cell region corresponding to the representative cell. Hereinafter, “image information about a cell region corresponding to the representative cell, positional information corresponding to the representative cell region, and characteristic information about a cell region corresponding to the representative cell” may be referred to as information about a representative cell.

The image information about a cell region corresponding to the representative cell includes an enlarged image of a small clipped region including at least the representative cell from the cell image acquired by the image acquisition unit 111. The characteristic information about the cell region corresponding to the representative cell includes measurement data on the representative cell obtained by measurements of the cell image. The measurement data on the representative cell is characteristic information such as morphological information and pixel information corresponding to the representative cell determined by the representative cell determination unit 120 in the cell data group acquired by the cell data acquisition unit 110.

If at least one of the pieces of information about the representative cell is selected, the display control unit 130 may control the display unit to display information other than selected information together with the selected information in synchronization and in a highlighted manner. Specifically, if at least one of the pieces of information about the representative cell displayed on the display is selected by a user operation, the display control unit 130 may control the display unit to display information different from the selected information in synchronization and in a highlighted manner.

According to the present exemplary embodiment, it is possible to determine a representative cell region (representative cell) from a cell image, and to cause the display unit to display the image of the representative cell and characteristic information. This enables a user to simply recognize information about the representative cell among a large number of cells within the cell image.

### <Cell Image Processing Method>

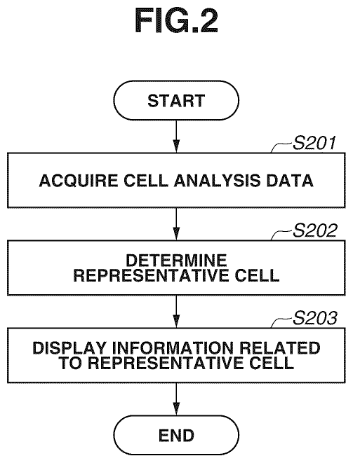

FIG. 2 is a flowchart illustrating a cell image processing method according to a second exemplary embodiment of the present disclosure. The cell image processing method according to the second exemplary embodiment includes a step of acquiring cell analysis data (step S201), a step of determining a representative cell (step S202), and a step of displaying information related to the representative cell (step S203).

Step S201 includes an image acquisition step of acquiring a cell image of a sample including a plurality of cells as a subject, and a cell region extraction step of extracting a plurality of cell regions respectively corresponding to cells from the cell image. Step S202 includes a representative cell determination step of determining a representative cell region from among the plurality of extracted cell regions based on at least one type of information constituting characteristic information. The term “characteristic information” as used herein refers to information that is acquired in the cell information acquisition step and includes morphological information about each of the cell regions and pixel information about each of the cell regions.

Step S203 includes a display control step of causing the display unit to display image information about a cell region corresponding to the representative cell, positional information corresponding to the representative cell region, and characteristic information about a cell region corresponding to the representative cell.

Each function of the cell image processing system 100 will be described in detail below with reference to the flowchart illustrated in FIG. 2.

### (Acquisition of Cell Data)

In step S201, the cell data acquisition unit 110 acquires a cell image and a cell image data group by performing an image analysis (image processing) on the cell image.

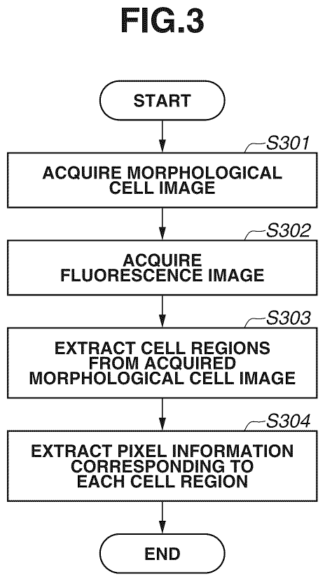

FIG. 3 is a flowchart illustrating processing for analyzing the cell image and acquiring cell data.

An example of processing for acquiring a defocused image and an autofluorescence image and acquiring cell data by image analysis will be described below with reference to FIG. 3.

### (Acquisition of Morphological Cell Image)

In step S301, the image acquisition unit 111 acquires a defocused image as a morphological cell image. As the image acquired in step S301, any image may be used as long as the image has a characteristic that the contrast of a contour portion of each cell is highlighted as compared with a non-cell region. For example, an image captured by a phase difference microscope, or an image captured using an oblique illumination system or a telecentric optical system that is telecentric at an object side and an image side (double telecentric optical system) can be used. A position where the defocused image is captured may be desirably a position where the contrast of the contour portion of each cell is highlighted most.

### (Acquisition of Fluorescence Image)

In step S302, the image data acquisition unit 101 acquires an autofluorescence image of each cell as a fluorescence image. The term “autofluorescence image” as used herein refers to an image obtained by irradiating a cell with excitation light with a predetermined wavelength to excite a cell-intrinsic fluorescent material and capturing an image of the fluorescence through a filter for cutting off the excitation light source wavelength.

A wavelength of excitation light and conditions for a cutoff filter may be selected depending on the fluorescence characteristics of an endogenous component to be observed. As a combination of a wavelength of excitation light and a cutoff filter, for example, when NAD (P) H is used as an endogenous component, excitation light with a wavelength of 360 nm and a long-pass filter of 430 nm are used. In the case of using flavins (FAD and the like), excitation light with a wavelength of 450 nm and a long-pass filter of 530 nm can be used. Other examples of the endogenous component to be used for autofluorescence observation include auto fluorescent molecules produced in a cell, such as collagen, fibronectin, tryptophan, and folic acid. Fluorescent endogenous components other than these components may also be used. The autofluorescence image is an image including the same field of view as that of the morphological cell image acquired in step S301. Further, any other image can be used as long as the image is captured by a camera including a two-dimensional image sensor in which two types or more of pixels having peak detection sensitivity in two or more different types of wavelength regions are regularly arranged.

For example, a red, green, and blue (RGB) color camera using a complementary metal-oxide semiconductor (CMOS) sensor as an optical sensor may be used.

However, the optical sensor according to the present exemplary embodiment is not limited to a CMOS sensor, and may be a camera using a charge-coupled device (CCD) sensor. It may be desirable to use an appropriate sensor or a camera using the sensor depending on the wavelength of light emitted from a cell to be observed.

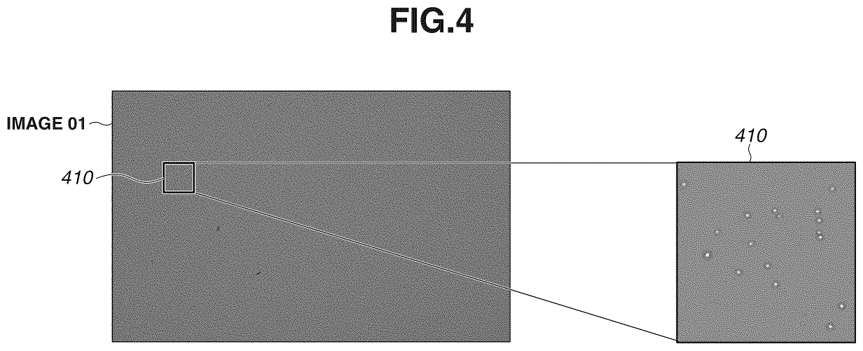

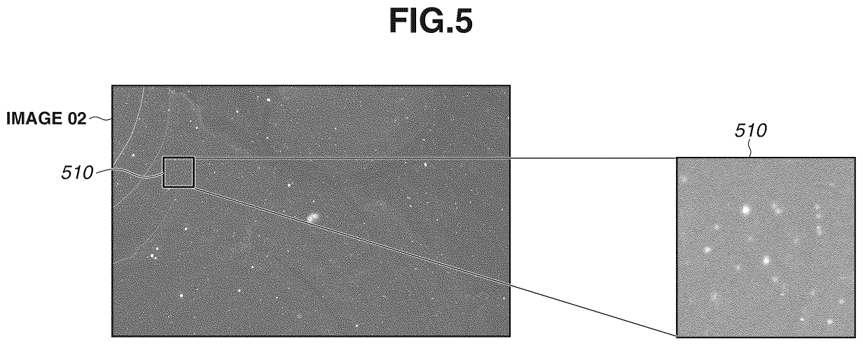

FIG. 4 illustrates an example of the defocused image acquired in step S301, and FIG. 5 illustrates an example of the autofluorescence image acquired in step S302. An image 01 illustrated in FIG. 4 is an example of the acquired defocused image, and an image 02 illustrated in FIG. 5 is an example of the autofluorescence image captured within the same field of view as that of the image 01 using excitation light with a wavelength of 360 nm and a long-pass filter of 430 nm. The image 01 and the image 02 are RGB color images captured by the RGB color camera using a CMOS sensor as an optical sensor.

An image 410 is an enlarged displayed image of a small region, which is a part of the image 01, and an image 510 is an enlarged displayed image of a small region, which is a part of the image 02. In the image 410, each region with a black highlighted contour corresponds to a cell region, and the image 410 is captured within the field of view in which approximately three thousand cells are present.

As the autofluorescence image, two or more autofluorescence images with different conditions for the combination of the wavelength of excitation light and observation-side cutoff filter characteristics may be acquired. The fluorescence image is not limited to an autofluorescence image, and a stained image obtained by observing a cell sample to which a staining reagent exhibiting fluorescence characteristics to a specific wavelength is added may be acquired.

### (Cell Region Extraction Processing)

In step S303, the cell region extraction unit 112 extracts each cell region from the morphological cell image acquired in step S301.

The process of extracting each cell region may be carried out by a known image analysis method. For example, in the case of the defocused image acquired as illustrated in FIG. 4, an RGB image acquired by the RGB color camera is converted into one channel and threshold processing is performed, thereby making it possible to extract cell contour information indicating a boundary between cell regions. Further, a mask image in which an internal region of the extracted cell contour is displayed with black pixels and an external region of the extracted cell contour is displayed with white pixels is created and an identifier is assigned to each isolated black-pixel region by labeling processing, thereby making it possible to identify each cell region in the cell image.

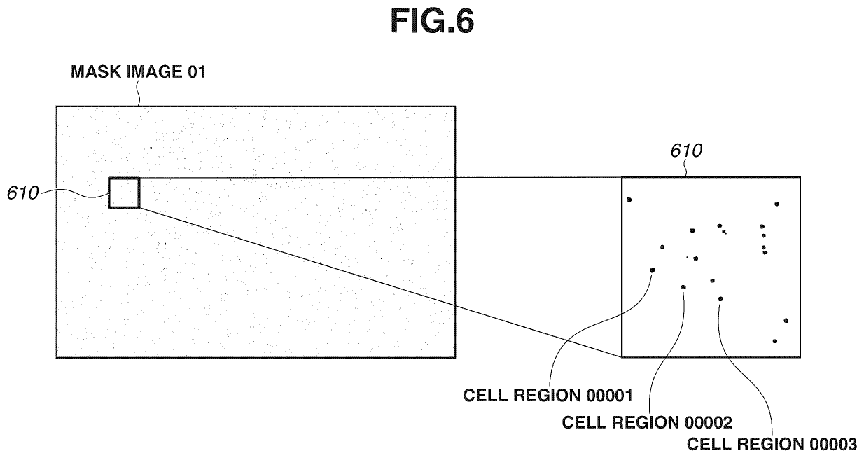

FIG. 6 illustrates an example of a mask image 01 created for the defocused image 01 illustrated in FIG. 4. An image 610 is an enlarged displayed image of a small region as a part of the mask image 01. Cell regions 00001 to 00003 each represent a partial cell region labeled based on the mask image 01.

While the present exemplary embodiment described above illustrates an example using the defocused image captured by the RGB color camera, the cell region extraction processing can be applied to an image in which the contrast of a contour portion of a cell is highlighted as compared with a non-cell region. For example, an image observed by a phase difference microscope, or an image on which image processing is performed to highlight the contour portion of each cell may be used.

### (Pixel Information Extraction Processing)

In step S304, the cell information acquisition unit 113 acquires pixel information corresponding to each cell region from the images acquired in step S301 and S302 based on each cell region acquired in step S303.

An example where an image is captured by the RGB color camera in step S302 will be described.

First, Bayer array data on the image is separated into images with three channels corresponding to RGB components, respectively. The Bayer array data is two-dimensional array data obtained by recording pixel values received by a CMOS color sensor.

Next, the pixel values of the Bayer array data are separated into components of color filters corresponding to three wavelength regions, respectively, thereby separating the components into three RGB components. In the Bayer array data, each pixel includes information about the pixel value corresponding to any one of the RGB components. Interpolation processing may be performed so that each pixel has information about the pixel values corresponding to the three RGB components, respectively. Further, pre-processing such as black-level correction processing for subtracting a dark current value from each pixel value, or filter processing for reducing noise may be performed.

Next, pixel information in each cell region acquired in step S303 is extracted. The pixel information is a scalar value indicating a pixel statistic of each of the RGB components in each cell region. For example, an average value is calculated as pixel information. An example where the average value is calculated as pixel value will be described below. However, the pixel information is not limited to this example, and one of an integrated value and a standard deviation, or a plurality of types of statistics may be calculated.

In the case of using a fluorescence image, the pixel statistic for only a significant component from among the RGB components may be calculated depending on the characteristics of the cutoff filter used during image capturing. For example, in the case of using an autofluorescence image captured using excitation light with a wavelength of 360 nm and a long-pass filter of 430 nm, the pixel statistic is calculated for only a G-component and a B-component.

The calculation of the pixel statistic as described above is repeatedly performed on each image and each cell region, thereby making it possible to acquire a cell data group as a set of pixel information about the cell region.

Further, morphological information indicating a morphological feature of each cell region may be acquired together with pixel information. Specifically, a scalar value such as an area, a diameter, a circularity, or a solidity is calculated.

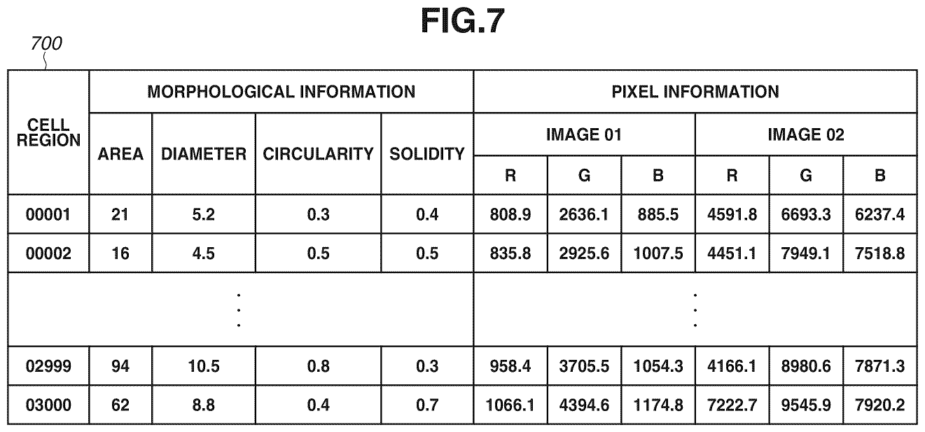

FIG. 7 illustrates an example of a cell data group acquired based on the image 01 and the image 02 illustrated in FIGS. 4 and 5, respectively, and the cell regions illustrated in FIG. 6. Table 700 is a table in which each row corresponds to each cell region and each column represents the result of calculation of pixel information and morphological information as described above.

In this case, the sum, ratio, or difference between two components of the three RGB components may be calculated and the calculated value may be used as pixel information about each cell region. For example, a calculation of (an average value of pixel values of G-component in the image 02)÷(an average value of pixel values of B-component in the image 02) is performed.

An example where the defocused image and the autofluorescence image are acquired in step S201 and cell data is acquired by image processing has been described above.

In step S201, the cell data acquisition unit 110 acquires a cell data group including both of a cell morphological feature and a cell functional feature. The acquired cell data group is used for processing of step S202. Positional information about each cell extracted in the cell image is also held in association with data on each cell region as illustrated in FIG. 7. Specifically, as the positional information about each cell, information about a group of coordinates of a contour surrounding each cell region and information about coordinates of a center of gravity are held.

While the present exemplary embodiment described above illustrates an example where a cell data group is acquired using the defocused image 01 and the autofluorescence image 02 captured within a specific field of view, the cell data group acquisition method is not limited to this example. Images captured in a plurality of fields of view may be acquired. In this case, the processing of step S201 is performed on each of images captured in the plurality of fields of view or on each of captured images of a plurality of cell samples, to thereby acquire the cell data group.

### (Determination of Representative Cell)

In step S202, the representative cell determination unit 120 determines a primary cell constituting the cell data group based on the cell data group acquired in step S201.

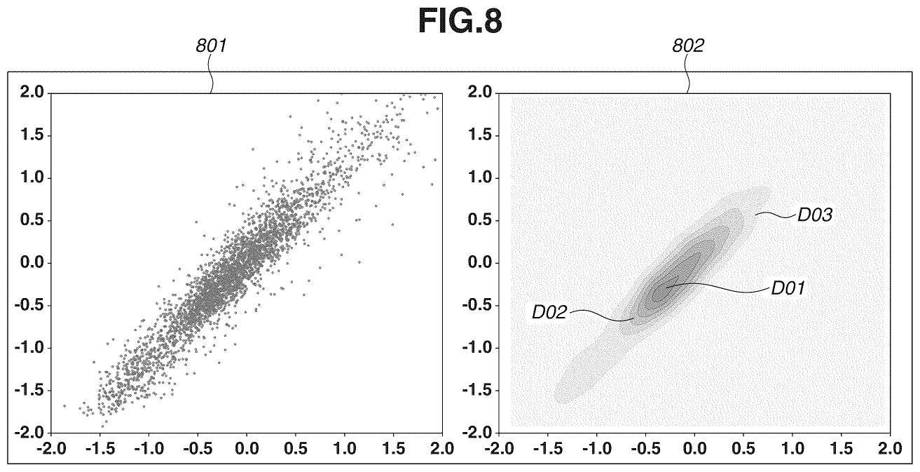

First, a probability density distribution is estimated based on the acquired cell data group. The probability density distribution is a distribution for estimating the probability at which certain data can occur. The probability density distribution is estimated by, for example, Kernel density estimation. FIG. 8 illustrates the result of estimation of the probability density distribution by Kernel density estimation using two types of information, i.e., the B-component of the image 02 and the G-component of the image 02 in the cell data group 700 illustrated in FIG. 7. A scatter diagram 801 is obtained by plotting standardized data on each cell region by taking the B-component of the image 02 on the horizontal axis and taking the G-component of the image 02 on the vertical axis. A probability density distribution 802 is a visualized probability density distribution estimated by Kernel density estimation on the data group indicated by the scatter diagram 801. A region with a darker color represents a region of data that occurs at a higher probability.

Next, in the probability density distribution 802, for example, a plurality of regions with a predetermined occurrence probability is set. For example, three regions with occurrence probabilities of 95 to 100%, 45 to 55%, and 0 to 5%, respectively, are set. The three regions correspond to regions D01, D02, and D03, respectively, in the probability density distribution 802. In this case, the region D01 is a region that occupies a majority of pieces of cell data and includes a large number of pieces of cell data on which the cell group to be observed is based. The region D02 includes a large number of cell groups in which a change in the morphological feature or the functional feature is likely to have occurred as compared with the cells included in the region D01. The region D03 is a region including cells that are highly likely to be in an unexpected state. Examples of cells in an unexpected state include cells that are transformed unexpectedly, cells that are contaminated with other cells, cells in an exceptional state dependent on a state (pathology) of the cells, and cell-like non-cellular objects that have occurred in an observation process. The region setting method is not limited to the above-described method. For example, two regions with occurrence probabilities of 95 to 100% and 0 to 5%, respectively, may be used and the occurrence probability range may be adjusted.

Next, data on the representative cell is determined from each of the regions D01 to D03. Specifically, first, the cell data group corresponding to the region D01 is extracted as a subset S01 of the cell data group 700 for the region D01. Further, several pieces of cell data are extracted from the subset S01 as a representative cell group G01 in the region D01. For example, four pieces of cell data may be extracted based on an unsupervised clustering method. As the cell data included in the representative cell group G01, cell data having a characteristic that is not similar to any other data in the subset may be extracted. For example, this processing may be implemented using an unsupervised clustering method. More specifically, the data group is divided into four clusters by the unsupervised clustering method such as K-Means, and data closest to the center of gravity of data in each cluster is determined to be representative cell data. The above-described procedure is also applied to each of the regions D02 and D03 to sequentially extract representative cell groups G02 and G03.

In step S202 described above, the representative cell groups G01, G02, and G03 are extracted as primary cells constituting the cell data group.

An example where a representative cell is determined using two types of data in the cell data group as illustrated in FIG. 7 has been described above with reference to step S202. However, the representative cell determination method is not limited to this example. The representative cell group can be determined in the same manner also in the case of using one or more types of data. For example, only diameter data or all types of data may be used. In particular, if a superior data type to represent cell properties is known among a plurality of data types, the data type may be desirably selected by the user and the representative cell may be desirably determined using the selected data type. If the superior data type is unknown, it is also effective to carry out the processing of step S202 after each N-dimensional cell data formed of N types of scalar values is converted into lower-dimensional data by a dimensional compression method such as a principal component analysis. For example, in the example illustrated in FIG. 7, each cell data is 10-dimensional data formed of ten types of items, and the principal component analysis is applied to the cell data group 700, thereby converting the data into two-dimensional data. Then, the representative cell is determined in step S202 using the two-dimensional data obtained after the conversion.

(Display of Information about Representative Cell)

In step S203, the display control unit 130 displays information about the representative cell extracted in step S202 on the display. As described above, information about the representative cell includes three types of information, i.e., a representative cell image, measurement data on the representative cell, and positional information about the representative cell.

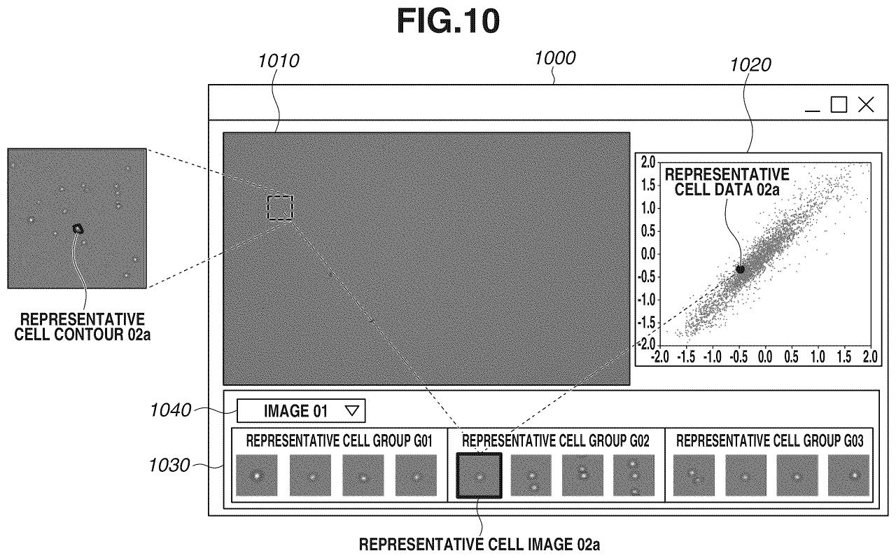

FIG. 10 illustrates an example of display of information about a representative cell. A graphical user interface (GUI) screen 1000 is displayed on the display. An area 1010 illustrates an example of the image acquired in step S201. For example, the defocused image 01 illustrated in FIG. 4 is displayed. Positional information about each representative cell extracted in step S202 is displayed on the defocused image 01 in a superimposed manner. As the positional information, for example, a contour as indicated by a representative cell contour 02a illustrated in FIG. 10 is drawn on the defocused image 01 based on contour coordinate information about the representative cell.

An area 1020 displays measurement data on each representative cell acquired in step S201. The measurement data on each representative cell may be desirably highlighted in consideration of an overview of the cell data group acquired in step S201. For example, the scatter diagram 801 illustrated in FIG. 8 is displayed and plotted dots respectively corresponding to the representative cells are highlighted. Highlighting is, for example, plotting data on each representative cell with a color different from that of data on a region other than the representative cell region, or plotting data on each representative cell with a marker size larger than that of data on a region other than the representative cell region. Information to be displayed on the area 1020 is not limited to a two-dimensional scatter diagram as illustrated in FIG. 10. Alternatively, a histogram, a box plot, a map obtained by visualizing the probability density distribution estimated in step S202, and the like may be used. Also, in such cases, data corresponding to each representative cell data is highlighted and drawn as a line or a dot.

An area 1030 displays representative cell images based on the positional information about each representative cell extracted in step S202. FIG. 10 illustrates an enlarged displayed image of clipped areas clipped out from the defocused image 01 illustrated in FIG. 4 and each having a predetermined size with the center of gravity of the representative cell as an image center.

The predetermined size may be determined by the user depending on the size of a cell to be observed. As indicated by the representative cell groups G01, G02, and G03 illustrated in FIG. 10, it may be desirable to display each representative cell group in such a manner that the user can recognize the region in the probability density distribution to which each representative cell belongs in step S202. The area 1010 and the area 1030 respectively correspond to the defocused image 01 and the representative cell image clipped from the defocused image 01. If a plurality of images is acquired as described above in step S201, the plurality of images can be switched. Specifically, the user selects an image displayed on a selected field 1040, thereby switching the image to be displayed on the area 1010 and the representative cell image displayed on the area 1030. The image switching processing is not limited to this example, and the images may be switched by a mouse operation or a keyboard operation.

The display control unit 130 displays information about each representative cell on the GUI screen. Then, when information about any one of the types of representative cells is selected by the user, the display control unit 130 controls the display unit to display information about other types of representative cells in synchronization and in a highlighted manner. For example, if a representative cell image 02a is selected by the user from among the representative cell images displayed on the area 1030 as illustrated in FIG. 10, the representative cell contour 02a and representative cell data 02a corresponding to the representative cell image 02a are highlighted.

A method for determining a representative cell in a cell data group acquired based on a cell image and displaying information about the representative cell has been described above with reference to steps S201 to S203. According to the present exemplary embodiment, it is possible for the user to simply recognize a primary representative cell constituting a cell data group.

The processing of steps S201 to S203 according to modified examples of the second exemplary embodiment will be described below. Steps that are not mentioned in the modified examples are similar to those of the second exemplary embodiment, and thus the descriptions thereof are omitted.

A first modified example of the second exemplary embodiment will be described. In step S302, if the morphological cell image and the fluorescence image are not captured in the same field of view, registration processing is performed on the morphological cell image and fluorescence image data.

The registration processing is performed by a method based on marking by an operator, or by a method using image processing.

In the method based on marking, the operator marks a plurality of points at the same cell positions in each of the morphological cell image and the autofluorescence image, and calculates an affine transform matrix based on the coordinates of the marked points, thereby performing the registration processing. In this case, nine or more points can be marked.

In the case of performing registration (alignment processing) by image processing, the alignment processing is performed using, for example, a mask image generated by threshold processing on an autofluorescence image, and a non-cell region mask image generated from the defocused image in step S302. In this case, a method such as template matching or phase only correlation may be used for alignment processing on two mask images.

According to the first modified example, even when the morphological cell image and the fluorescence image are not captured in the same field of view, cell data including both of the cell morphological feature and the cell functional feature can be acquired.

### Second Modified Example of Second Exemplary Embodiment

A second modified example of the second exemplary embodiment will be described. The second exemplary embodiment described above illustrates an example of acquiring a morphological image and a fluorescence image as an example of acquiring cell data including both of the morphological feature and the functional feature of each cell. The second exemplary embodiment is also applicable to a case where only the morphological image or the fluorescence image is used as an observation image of a target cell sample.

In the case of using only the morphological image, the fluorescence image is not acquired in step S302 according to the second exemplary embodiment, and the processing proceeds to step S303. The subsequent processing can be implemented in the same manner as in the second exemplary embodiment.

In the case of using only the fluorescence image, the morphological image is not acquired in step S301 according to the second exemplary embodiment, and the processing proceeds to step S302. To extract each cell region in step S303, a known image processing method may be used. For example, mask image creation processing and labeling processing as illustrated in FIG. 6 are performed by threshold processing after the fluorescence image is converted into a grayscale image.

A threshold used in the threshold processing may be determined by a known method such as Otsu's method. The subsequent processing can be performed in the same manner as in the second exemplary embodiment described above.

A third modified example of the second exemplary embodiment will be described. In step S304, the method for extracting RGB components from Bayer array data is described. However, the components may be extracted after the RGB components are converted into different color spaces or different color coordinate systems. For example, color spaces such as sRGB and AdobeRGB and color coordinate systems such as Lab, XYZ, and HSV can be used.

A fourth modified example of the second exemplary embodiment will be described. The second exemplary embodiment described above illustrates an example of the autofluorescence information extraction method using a CMOS sensor as an RGB camera. Alternatively, an image acquired by a camera other than an RGB camera, such as a multiband spectral camera including an optical sensor suitable for an observation wavelength, or a spectral camera such as a hyper spectral camera may be used. The optical sensor is, for example, a CMOS sensor, a CCD sensor, a Ge-type sensor, an InGaAs-type sensor, a bolometer-type sensor, or an avalanche photodiode array (APD)-type sensor.

In this case, in step S304, the image data is separated into images of a plurality of components corresponding to a plurality of wavelength filters included in the camera.

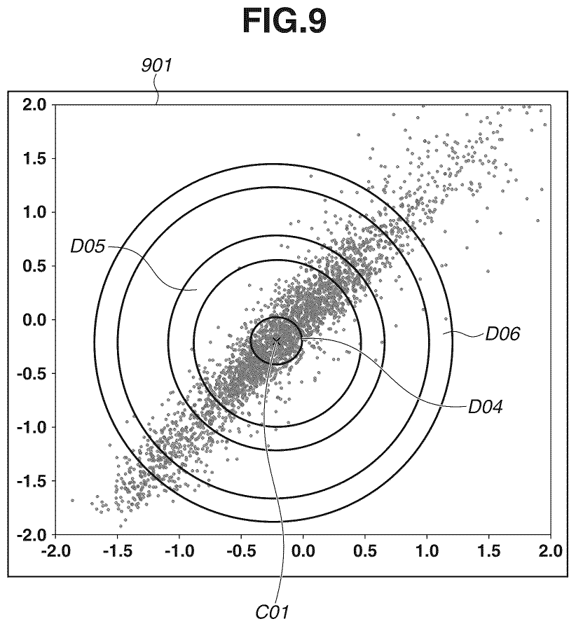

A fifth modified example of the second exemplary embodiment will be described. The estimation of a probability density distribution according to the second exemplary embodiment is effective when a dense cell data distribution is present in a data space formed of the type of data used for estimation. On the other hand, in a cell data group in which a dense cell data distribution is not present, a representative cell group may be determined based on a distance of data from the center of gravity of the cell data group. Specifically, a plurality of data distribution regions is set based on the distance of data from the center of gravity of the cell data group. The graph 901 in FIG. 9 illustrates an example of setting regions, for example, in the scatter diagram 801 illustrated in FIG. 8. Three regions D04, D05, and D06 corresponding to distance ranges of 0.00 to 0.10, 0.45 to 0.55, and 0.95 to 1.00, respectively, are set with respect to a center of gravity C01 of the cell data group. The distance from the center of gravity is normalized to 1.00 at maximum.

A method for determining the representative cell in each of the regions D04, D05, and D06 is similar to that of the second exemplary embodiment.

Which one of the estimations of a probability density distribution according to the second exemplary embodiment and the method according to the present modified example is used may be determined using a known accuracy evaluation index for estimation of the probability density distribution. Specifically, data occurrence probabilities are calculated and the total value of the calculated data occurrence probabilities can be used as the value of the accuracy evaluation index. The evaluation value is a value in a range from 0.0 to 1.0. For example, if the evaluation value is less than or equal to 0.8, the method is switched to the determination of the representative cell based on the present modified example.

The scatter diagram 801 illustrated in FIG. 8 or a map representing the result of the probability density distribution 802 may be displayed and the method to be employed may be switched after the result is checked by the user.

A sixth modified example of the second exemplary embodiment will be described. The second exemplary embodiment described above illustrates an example of displaying representative cell images in a list format as illustrated in FIG. 10. However, at least two types of information among three types of information, i.e., a representative cell image, positional information about the representative cell, and measurement data on the representative cell may be displayed, and a format for displaying such information is not limited to the example illustrated in FIG. 10.

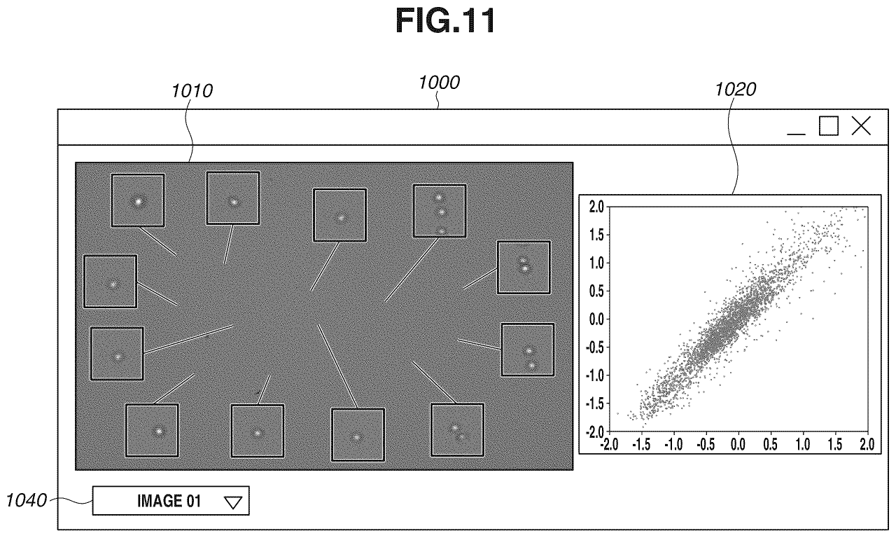

For example, the display control unit may cause the display unit to display image information about the cell region corresponding to the representative cell and the position of the representative cell region in the cell image in an associated manner. Specifically, a format in which the representative cell image is mapped and displayed at the coordinates on the image displayed on the area 1010, or at the position corresponding to the data on the scatter diagram illustrated in area 1020 may be used. FIG. 11 illustrates an example of mapping and displaying representative cell images based on the coordinates on the area 1010. Each representative cell image is displayed on the image 01 on the area 1010 and each representative cell is displayed in association with the position of center of gravity of the corresponding representative cell.

Instead of displaying the representative cell images on the same GUI screen 1000, the representative cell images may be displayed on the areas 1010, 1020, and 1030, respectively, or on one of the areas 1010, 1020, and 1030 as a separate GUI screen.

The display formats described in the second exemplary embodiment and the present modified example may be arbitrarily switched by the user. For example, an appropriate display format can be selected depending on the type of a device used for display, such as a desktop personal computer (PC), a laptop PC, a tablet PC, or a smartphone, and the resolution of the display.

The second exemplary embodiment described above illustrates a method for determining a representative cell in the cell data group acquired based on the cell image and displaying information about the representative cell.

A third exemplary embodiment illustrates a method for performing cluster classification by unsupervised clustering on the acquired cell data group to determine the representative cell in each cluster.

According to the third exemplary embodiment, primary cell data constituting each cell data group can be appropriately determined to be a representative cell even in a case where a cell sample to be analyzed includes a large number of cell groups with different properties.

The third exemplary embodiment is especially effective when it can be estimated in advance that a target cell sample includes cell groups with different properties. Examples of such a case include an image obtained by observing a cell sample in which cell are cocultured by adding two or more different types of cell stains or two or more different types of reagents, and a cell sample in a state where a large number of living cells and a large number of dead cells are present.

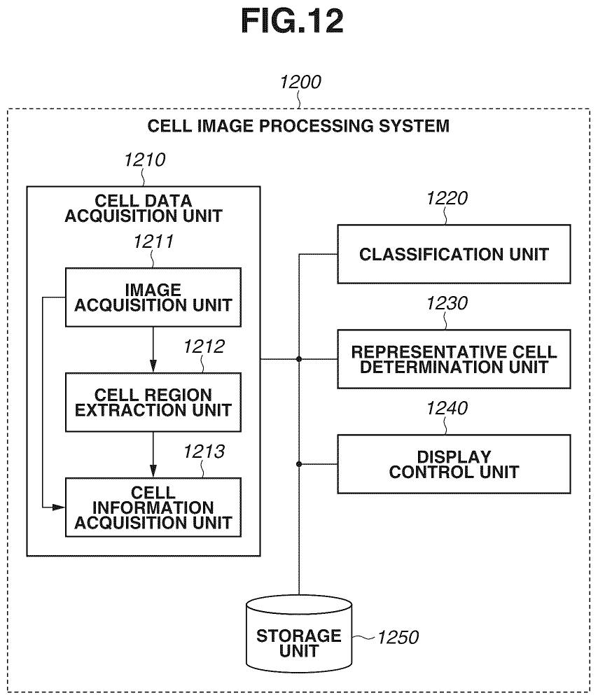

FIG. 12 is a block diagram illustrating functional blocks of a cell image processing system according to the third exemplary embodiment. A cell image processing system 1200 includes a cell data acquisition unit 1210, a classification unit 1220, a representative cell determination unit 1230, a display control unit 1240, and a storage unit 1250. The cell data acquisition unit 1210 includes an image acquisition unit 1211, a cell region extraction unit 1212, and a cell information acquisition unit 1213.

The cell data acquisition unit 1210, the display control unit 1240, the image acquisition unit 1211, the cell region extraction unit 1212, and the cell information acquisition unit 1213 are similar to those of the first exemplary embodiment, and thus the descriptions thereof are omitted.

### (Classification Unit)

The classification unit 1220 classifies the cell data group acquired by the cell data acquisition unit 1210 into a plurality of clusters by an unsupervised clustering method.

### (Representative Cell Determination Unit)

The representative cell determination unit 1230 determines representative cell data, which is primary cell data constituting each cluster in each cell data group in each of the clusters classified by the classification unit 1220.

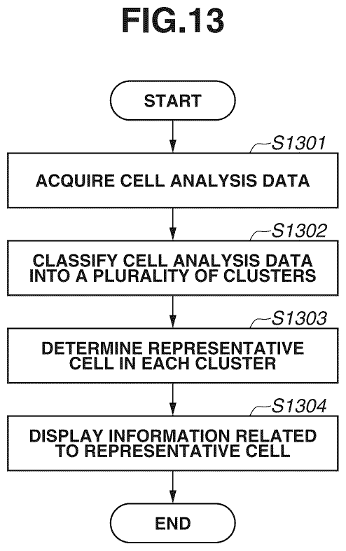

FIG. 13 is a flowchart illustrating processing to be performed by a cell image processing system according to the third exemplary embodiment. Each function of the cell image processing system 1200 will be described in detail below with reference to the flowchart illustrated in FIG. 13.

Step S1301 is similar to step S201 according to the second exemplary embodiment, and thus the description thereof is omitted.

### (Cluster Classification)

In step S1302, the classification unit 1220 classifies the cell data group acquired in step S1301 into a plurality of clusters.

For example, the cell data group is classified into two clusters CL01 and CL02 by an unsupervised clustering method such as K-Means using the number of clusters “2”. In this case, the number of clusters can be set to an arbitrary number by the user. For example, in the case of a cell image of a cell sample in which two different types of cell stains are cocultured, two clusters are set.

### (Determination of Representative Cell)

In step S1303, the representative cell determination unit 1230 determines the representative cell in each cell data group belonging to the corresponding cluster based on the classification result in step S1302. Like in the second exemplary embodiment, as a method for determining the representative cell in each cluster, a probability density distribution may be estimated based on the cell data group belonging to each cluster, and subsets and representative cell data may be extracted based on the data occurrence probability.

In the classification result, only a small number of pieces of cell data may belong to a specific classification result in some cell samples. For example, the number of cells belonging to a specific cluster may be less than 100. In such a case, it may be desirable not to estimate the probability density distribution in the second exemplary embodiment, and it may be desirable not to set predetermined regions, such as the regions D01 and D02 based on the estimation of the probability density distribution is performed, and it may also be desirable not to extract subsets respectively corresponding to the regions. Alternatively, all data belonging to clusters may be treated as a data group equivalent to a subset according to the second exemplary embodiment and representative cell data may be determined based on the data.

For example, if a cell data group is classified into two clusters A and B and regions with occurrence probabilities of 95 to 100% and 45 to 55%, respectively, are set, representative cell groups A01 and A02 of the cluster A and representative cell groups B01 and B02 of the cluster B are extracted as representative cells.

In a case where a cell data group is classified into a plurality of clusters and representative cells constituting each cluster are determined as in the present exemplary embodiment, cell data present in the vicinity of a boundary between different clusters is also important cell data for the user to recognize the state of each cell. For example, if a cell image of a cell sample in which two different types of cell stains are cocultured is classified into two clusters, data present in the vicinity of a boundary between the clusters has similar characteristics among the different types of cells. Accordingly, it is highly likely that the data may be important for the user in analyzing cell data.

Accordingly, in the present exemplary embodiment, data present in the vicinity of the boundary between clusters is also extracted as representative cell data. Specifically, an occurrence probability on the probability density distribution estimated based on a cell data group in another cluster is calculated for cell data belonging to any cluster, and cell data groups with a certain occurrence probability or higher are extracted as subsets. Then, representative cell data is determined from the subsets by a method similar to that of the second exemplary embodiment. For example, if a cell image is classified into two clusters of clusters A and B and probability density distributions DA and DB are estimated for the clusters A and B, respectively, the occurrence probability on the probability density distribution DB of cell data belonging to the cluster A is calculated. Further, data groups with an occurrence probability of 50% or more are extracted as subsets. Four pieces of representative cell data are extracted as the representative cell data group from the subsets.

An identifier, such as a representative cell group A03b, is assigned so that the user can distinguish the extracted representative cell group from representative cell groups extracted by the method according to the second exemplary embodiment and the user can distinguish the probability density distribution in any one of the clusters based on which the representative cell group is extracted. Also, in the cluster B, a representative cell group B03a is extracted based on the probability density distribution DA.

(Display of Information about Representative Cell)

In step S1304, the display control unit 1240 displays information about the representative cells in each cluster extracted in step S1303 on the display.

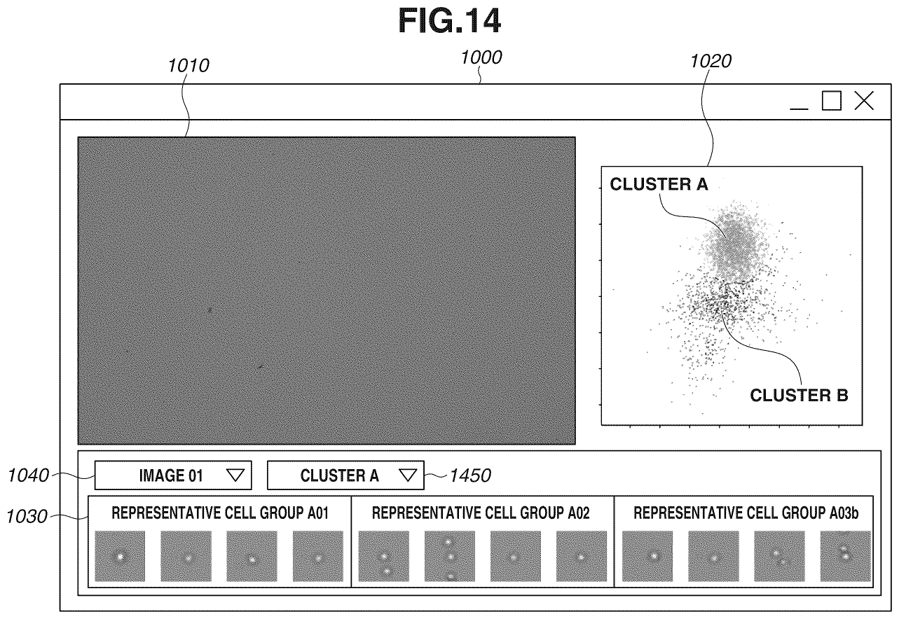

A representative cell image in each cluster may be displayed in a list format in which the area 1030 illustrated in FIG. 10 is enlarged and each row represents the representative cell image in each cluster. Like an area 1450 illustrated in FIG. 14, a UI for selecting a cluster to be displayed may be provided to switch the representative cell image to be displayed on the area 1030 based on the selected cluster.

As described above, according to the present exemplary embodiment, even when a cell sample to be analyzed includes a large number of cell groups with different properties, primary cell data constituting a cell data group can be appropriately determined to be a representative cell.

As described above, the present exemplary embodiment is effective when it can be estimated in advance that a target cell sample includes cell groups with different properties. However, if it is unknown whether a target cell sample includes cell groups with different properties, an example in which the second exemplary embodiment and the third exemplary embodiment are sequentially carried out is also effective. For example, the user determines the representative cell and checks the display result according to the second exemplary embodiment as illustrated in FIG. 10, and then determines whether the cell sample includes a large number of cell groups with different properties. As a result, if the cell sample includes cell groups with different properties, the representative cell determination processing including classification into clusters according to the third exemplary embodiment is carried out.

### <Program>

A program according to the present exemplary embodiment is a program for causing a computer to execute a cell image processing method according to the present exemplary embodiment described above.

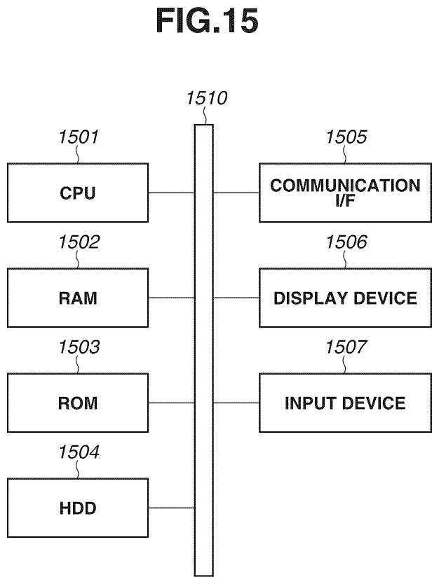

FIG. 15 is a block diagram illustrating a hardware example of an information processing system configured to execute the program according to the present exemplary embodiment.

The information processing system includes functions of a computer. For example, the information processing system may be integrally formed with a desktop PC, a laptop PC, a tablet PC, a smartphone, or the like.

To implement the functions as a computer for performing arithmetic processing and storage processing, the information processing system includes a central processing unit (CPU) 1501, a random access memory (RAM) 1502, a read-only memory (ROM) 1503, and an HDD 1504. The information processing system also includes a communication I/F 1505, a display device 1506, and an input device 1507. The CPU 1501, the RAM 1502, the ROM 1503, the HDD 1504, the communication I/F 1505, the display device 1506, and the input device 1507 are interconnected via a bus 1510. The display device 1506 and the input device 1507 may be connected to the bus 1510 via a drive device (not illustrated) for driving these devices.

While FIG. 15 illustrates the units constituting the information processing system as an integrated device, some of these functions may be configured as an external device. For example, the display device 1506 and the input device 1507 may be external devices separate from a portion constituting the functions of the computer including the CPU 1501.

The CPU 1501 includes functions for performing a predetermined operation based on programs stored in the RAM 1502, the HDD 1504, or the like, and controlling each unit of the information processing system. The RAM 1502 is composed of a volatile storage medium, and provides a temporary memory area for operation of the CPU 1501. The ROM 1503 is composed of a nonvolatile storage medium, and stores required information such as programs used for operation of the information processing system. The HDD 1504 is a storage device that is composed of a nonvolatile storage medium and stores information about the number of individual separated sections and positions, a fluorescence intensity, and the like.

The communication I/F 1505 is a communication interface based on standards such as Wi-Fi® or 4G, and is a module for communicating with another device. The display device 1506 is a liquid crystal display, an organic light emitting diode (OLED) display, or the like, and is used to display moving images, still images, characters, and the like. The input device 1507 is a button, a touch panel, a keyboard, a pointing device, or the like, and is used for the user to operate the information processing system. The display device 1506 and the input device 1507 may be integrally formed as a touch panel.

The hardware illustrated in FIG. 15 is merely an example. Devices other than the above-described devices may be added and some of the above-described devices may be omitted. Some of the devices may be replaced by other devices including similar functions. Further, some of the functions may be provided by another device via a network, and the functions constituting the present exemplary embodiment may be implemented by being distributed to a plurality of devices. For example, the HDD 1504 may be replaced by a solid-state drive (SSD) using a semiconductor element such as a flash memory, or may be replaced by a cloud storage.

The above-described present disclosure includes the following examples, method, program, and storage medium.

### Example 1

There is provided a cell image processing system including: an image acquisition unit configured to acquire a cell image of a sample including a plurality of cells as a subject; a cell region extraction unit configured to extract a plurality of cell regions respectively corresponding to cells from the cell image; a cell information acquisition unit configured to acquire characteristic information including morphological information about each of the cell regions and pixel information about each of the cell regions; a representative cell determination unit configured to determine a representative cell region from among the plurality of extracted cell regions based on at least one type of information constituting the characteristic information; and a display control unit configured to cause a display unit to display image information about a cell region corresponding to the representative cell, positional information corresponding to the representative cell region, and characteristic information about a cell region corresponding to the representative cell.

### Example 2

The cell image processing system according to Example 1 is provided, in which the positional information includes at least one of a group of contour coordinates indicating a boundary between cell regions in the cell image, and coordinates indicating a position of center of gravity of a cell region in the cell image.

### Example 3

The cell image processing system according to Example 1 or 2 is provided, in which the morphological information about each of the cell regions includes at least one of an area of the cell region, a diameter of the cell region, a circularity of the cell region, and a perimeter of a contour of the cell region.

### Example 4

The cell image processing system according to any one of Examples 1 to 3 is provided, in which the pixel information about each of the cell regions includes at least one of an average value of pixel values in the cell region, an integrated value of pixel values in the cell region, and a standard deviation of pixel values in the cell region.

### Example 5

The cell image processing system according to any one of Examples 1 to 4 is provided, in which the cell image includes at least one of a fluorescence image, a bright-field image, and a phase difference image.

### Example 6

The cell image processing system according to any one of Examples 1 to 5 is provided, in which the representative cell determination unit extracts a plurality of subsets from a data group formed of the characteristic information about the plurality of cell regions and determines the representative cell region from each of the plurality of subsets.

### Example 7

The cell image processing system according to Example 6 is provided, in which the representative cell determination unit calculates a probability of occurrence of data on each cell region based on at least one type of information constituting the characteristic information, and extracts, as a subset, a data group corresponding to each of a plurality of predetermined occurrence probability ranges from the data group formed of the characteristic information about the plurality of cell regions.

### Example 8

The cell image processing system according to Example 6 is provided, in which the representative cell determination unit calculates a distance of data on each cell region from a center of gravity of data on all the cell regions based on at least one type of information constituting the characteristic information, and extracts, as a subset, a data group corresponding to each of a plurality of predetermined distance ranges from the data group formed of the characteristic information about the plurality of cell regions.

### Example 9

The cell image processing system according to any one of Examples 6 to 8 is provided, in which the representative cell determination unit divides the data group constituting the subsets into a plurality of clusters by unsupervised clustering, and determines a cell region corresponding to data closest to a position of center of gravity of the data group to be the representative cell region in each of data groups respectively corresponding to the clusters.

### Example 10

The cell image processing system according to any one of Examples 1 to 9 is provided, further including a classification unit configured to classify a cell data group constituting the cell image into a plurality of clusters, in which the representative cell determination unit determines the representative cell region in each of the plurality of clusters.

### Example 11

The cell image processing system according to any one of Examples 1 to 10 is provided, in which, in a case where any one of image information about a cell region corresponding to the representative cell, positional information corresponding to the representative cell region, and characteristic information about a cell region corresponding to the representative cell is selected, the display control unit controls the display unit to display information other than the selected information together with the selected information in synchronization and in a highlighted manner.

### Example 12

The cell image processing system according to any one of Examples 1 to 10 is provided herein, in which the display control unit controls the display unit to display image information about a cell region corresponding to the representative cell and a position of the representative cell region in the cell image in an associated manner.

### (Method)

The present disclosure provides a cell image processing method including: an image acquisition step of acquiring a cell image of a sample including a plurality of cells as a subject; a cell region extraction step of extracting a plurality of cell regions respectively corresponding to cells from the cell image; a cell information acquisition step of acquiring characteristic information including morphological information about each of the cell regions and pixel information about each of the cell regions; a representative cell determination step of determining a representative cell region from among the plurality of extracted cell regions based on at least one type of information constituting the characteristic information; and a display control step of controlling a display unit to display image information about a cell region corresponding to the representative cell, positional information corresponding to the representative cell region, and characteristic information about a cell region corresponding to the representative cell.

### (Program)

A program for causing a computer to execute a cell image processing method according to the method is provided herein.

### (Non-Transitory Computer Readable Storage Medium)

A storage medium storing a program for causing a computer to execute a cell image processing method according to the method is also provided.

According to an aspect of the present disclosure, it is possible to extract a primary cell (representative cell) from a large number of cells even when a cell image to be analyzed includes a large number of cells, and it is also possible to display the extracted representative cell together with information about the representative cell.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2023-201982, filed Nov. 29, 2023, which is hereby incorporated by reference herein in its entirety.

What is claimed is:

1. A cell image processing system comprising:
an image acquisition unit configured to acquire a cell image of a sample including a plurality of cells as a subject;
a cell region extraction unit configured to extract a plurality of cell regions respectively corresponding to cells from the cell image;
a cell information acquisition unit configured to acquire characteristic information including morphological information about each of the cell regions and pixel information about each of the cell regions;
a representative cell determination unit configured to determine a representative cell region from among the plurality of extracted cell regions based on at least one type of information constituting the characteristic information; and
a display control unit configured to cause a display unit to display image information about a cell region corresponding to the representative cell, positional information corresponding to the representative cell, and characteristic information about a cell region corresponding to the representative cell.

2. The cell image processing system according to claim 1, wherein the positional information includes at least one of a group of contour coordinates indicating a boundary between cell regions in the cell image, and coordinates indicating a position of center of gravity of a cell region in the cell image.

3. The cell image processing system according to claim 1, wherein the morphological information about each of the cell regions includes at least one of an area of the cell region, a diameter of the cell region, a circularity of the cell region, and a perimeter of the cell region.

4. The cell image processing system according to claim 1, wherein the pixel information about each of the cell regions includes at least one of an average value of pixel values in the cell region, an integrated value of pixel values in the cell region, and a standard deviation of pixel values in the cell region.

5. The cell image processing system according to claim 1, wherein the cell image includes at least one of a fluorescence image, a bright-field image, and a phase contrast image.

6. The cell image processing system according to claim 1, wherein the representative cell determination unit extracts a plurality of subsets from a data group formed of the characteristic information about the plurality of cell regions and determines the representative cell region from each of the plurality of subsets.

7. The cell image processing system according to claim 6, wherein the representative cell determination unit calculates a probability of occurrence of data on each cell region based on at least one type of information constituting the characteristic information, and extracts, as a subset, a data group corresponding to each of a plurality of predetermined occurrence probability ranges from the data group formed of the characteristic information about the plurality of cell regions.

8. The cell image processing system according to claim 6, wherein the representative cell determination unit calculates a distance of data on each cell region from a center of gravity of data on all the cell regions based on at least one type of information constituting the characteristic information, and extracts, as a subset, a data group corresponding to each of a plurality of predetermined distance ranges from the data group formed of the characteristic information about the plurality of cell regions.

9. The cell image processing system according to claim 6, wherein the representative cell determination unit divides the data group constituting the subsets into a plurality of clusters by unsupervised clustering, and determines a cell region corresponding to data closest to a position of center of gravity of the data group to be the representative cell region in each of data groups respectively corresponding to the clusters.

10. The cell image processing system according to claim 1, further comprising a classification unit configured to classify a cell data group constituting the cell image into a plurality of clusters,
wherein the representative cell determination unit determines the representative cell region in each of the plurality of clusters.

11. The cell image processing system according to claim 1, wherein, in a case where any one of image information about a cell region corresponding to the representative cell, positional information corresponding to the representative cell region, and characteristic information about a cell region corresponding to the representative cell is selected, the display control unit controls the display unit to display information other than the selected information together with the selected information in synchronization and in a highlighted manner.

12. The cell image processing system according to claim 1, wherein the display control unit controls the display unit to display image information about a cell region corresponding to the representative cell and a position of the representative cell region in the cell image in an associated manner.

13. A cell image processing method comprising:
acquiring a cell image of a sample including a plurality of cells as a subject;
extracting a plurality of cell regions respectively corresponding to cells from the cell image;
acquiring characteristic information including morphological information about each of the cell regions and pixel information about each of the cell regions;
determining a representative cell region from among the plurality of extracted cell regions based on at least one type of information constituting the characteristic information; and
controlling a display unit to display image information about a cell region corresponding to the representative cell, positional information corresponding to the representative cell, and characteristic information about a cell region corresponding to the representative cell.

14. A non-transitory computer readable storage medium storing a program for causing a computer to execute a cell image processing method according to claim 13.
